(19) United States
(12) Patent Application Publication
(10) Pub. No.: US 2023/0210782 A1
(43) Pub. Date: Jul. 6, 2023

(54) NON-PORCINE FORMULATIONS AND METHODS THEREOF

(71) Applicant: AZURRX BIOPHARMA, INC., Brooklyn, NY (US)

(72) Inventors: Dinesh Srinivasan, San Jose, CA (US); James Pennington, Lafayette, CA (US); Ted Stover, Hobe Sound, FL (US); Mathieu Schue, Saint-Laurent-des-Arbres (FR)

(21) Appl. No.: 18/009,619

(22) PCT Filed: Jun. 17, 2021
(86) PCT No.: PCT/US2021/037850

§ 371 Date: Dec. 9, 2022

(30) Foreign Application Priority Data
Jun. 18, 2020 (FR) 2006394

(51) Int. Cl. A61K 9/48; A61K 38/46; A61K 45/06; A61K 9/00; A61P 1/18

(52) U.S. Cl. CPC A61K 9/4891; A61K 38/465; A61K 45/06; A61K 9/0053; A61P 1/18

(57) ABSTRACT

Disclosed are compositions and formulations of non-porcine lipase and methods of treatment and manufacture thereof.

### FIELD OF THE INVENTION

The present invention relates to non-porcine lipase composition and dosage forms, methods of treatment and methods of preparation.

Lipase 2 (Lip2) from Yarrowia lipolytica (i.e., MS1819) is an autologous yeast recombinant lipase. The targeted indication of MS1819 is the compensation of exocrine pancreatic insufficiency (EPI) due to cystic fibrosis, chronic pancreatitis and other indications the exocrine pancreas is responsible for. The symptomatology of EPI is essentially due to pancreatic lipase deficiency, an enzyme that hydrolyses triglycerides into monoglycerides and free fatty acids.

Chronic Pancreatitis (CP), the most common cause of EPI, is a long-standing inflammation of the pancreas that alters its normal structure and functions, which is associated with EPI in about 60% of patients. Cystic fibrosis (CF), another frequent aetiology of EPI, is a severe genetic disease associated with chronic morbidity and life-span decrease of most affected individuals. About 80-90% of patients with CF develop EPI. In addition, EPI is common after surgical resection of the pancreas, which is usually performed as a result of cancer or complications of CP. Other less common aetiologies of EPI include gastric surgery, certain intestinal disorders (e.g. severe celiac disease, small bowel resection, and enteral-artificial nutrition), and pancreatic diseases (e.g. pancreatic trauma, severe acute pancreatitis with pancreatic necrosis, and pancreatic cancer).

The compensation of EPI, which classically relies on porcine pancreatic extracts (PPEs, also referred to as porcine pancreatic replacement therapy (PERT)), has been a concern of the Food and Drug Administration (FDA) because of the animal ingredients used for the preparation of PPEs and the related risk of transmission of conventional and non-conventional infectious agents. In addition, the dose of PPE that can be given may be limited, especially in CF, due to the risk of fibrosing colonopathy possibly associated with the presence of proteases and/or gastro-protection agents.

Accordingly, there is currently a need in the art for compositions and dosage forms for the treatment of EPI.

### SUMMARY OF THE INVENTION

The present invention is directed to non-porcine lipase dosage forms, methods of treatment and methods of manufacture.

In certain embodiments, the invention is directed to a delayed release oral dosage form comprising a non-porcine lipase and an enteric material (e.g., coating or dispersed with the active).

In certain embodiments, the invention is directed to spray dried non-porcine lipase compositions (with or without an enteric material).

In certain embodiments, the invention is directed to a dosage form comprising a non-porcine lipase; and a second active agent selected from a fat-soluble vitamin, a protease, an amylase, a porcine pancreatic enzyme replacement, other non-porcine replacements, or a combination thereof.

In certain embodiments, the invention is directed to processes for manufacturing the compositions and dosage forms disclosed herein.

In certain embodiments, the invention is directed to methods of treating exocrine pancreatic insufficiency comprising administering a dosage form as disclosed herein. In certain embodiments the insufficiency can be caused by one or more of acute or chronic pancreatitis, cystic fibrosis, pancreatectomy (associated with or without cancer such as pancreatic cancer), age related, Shwachman-Diamond Syndrome, diabetes type 1, diabetes type 2, HIV, celiac disease, or inflammatory bowel disease (such as ulcerative colitis or Crohn's disease).

The present invention is also directed in certain embodiments to a method of treating a condition (e.g., colon disease or other condition that is treatable by targeted administration of an active agent to the colon) by administering to a subject any of the compositions disclosed herein. The delivery can be to treat a colon disease or condition and can also be used to treat a systemic condition with a drug that is suitable for absorption in the colon.

The present invention is also directed in certain embodiments to a method of delivering an active agent to the colon of a patient by orally administering any of the delayed release formulations disclosed herein. In certain embodiments, at least 80%, at least 85%, at least 90%, at least 95%, or at least 99% of the active agent is delivered to the colon of the patient.

### BRIEF DESCRIPTION OF THE DRAWINGS

FIG. 1 is a graphical representation of the results of Example 3.

FIG. 2 is a representation of the clinical study of Example 4.

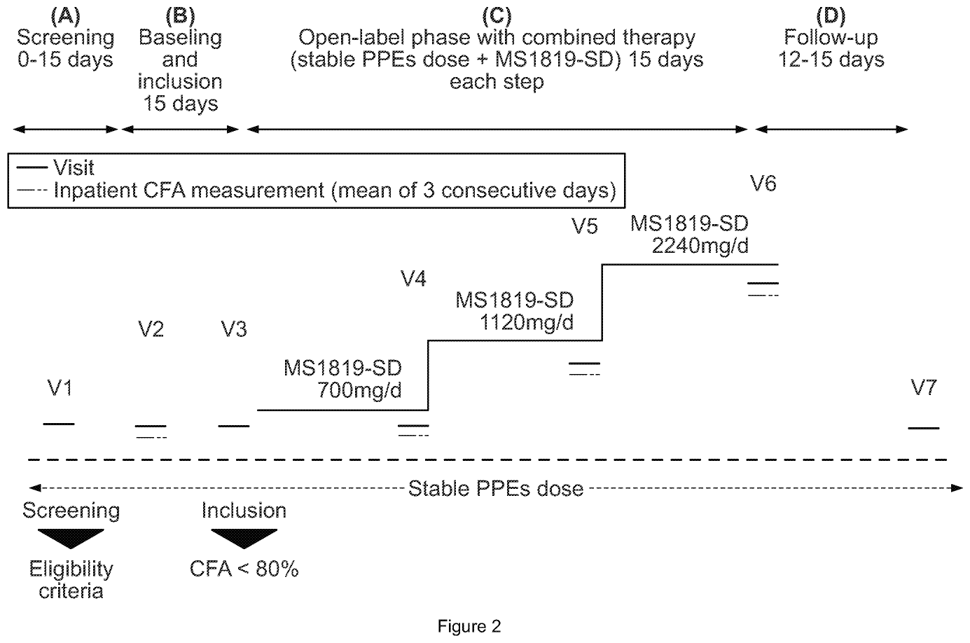

### DETAILED DESCRIPTION OF THE INVENTION

The present invention advances the state of the art by developing non-porcine lipase compositions, dosage forms, methods of treatment and methods of preparation.

The present invention advances the state of the art by developing non-porcine lipase compositions, dosage forms, methods of treatment and methods of preparation.

In certain embodiments, the present invention is directed to a delayed or immediate or sustained release oral dosage form comprising a non-porcine lipase. In alternative embodiments, the dosage form comprises an enteric material encompassing or dispersed with the non-porcine lipase.

In certain embodiments, the invention is directed to spray dried non-porcine lipase as disclosed herein.

In certain embodiments, the invention is directed to dosage form comprising (a) a non-porcine lipase; and (b) a second active agent selected from a fat-soluble vitamin, a protease, an amylase, a porcine pancreatic enzyme replacement, other non-porcine replacements, or a combination thereof. In certain embodiments, the vitamin is A, D, E, K or combinations thereof. In other embodiments, the second active agent is pancrelipase, liprotamase or a combination thereof

In certain embodiments, the invention is directed to dosage form comprising (a) a non-porcine lipase; and (b) a second active agent selected from a fat-soluble vitamin, a protease, an amylase, a porcine pancreatic enzyme replacement, other non-porcine replacements, or a combination thereof. In certain embodiments, the vitamin is A, D, E, K or combinations thereof. In other embodiments, the second active agent is pancrelipase, liprotamase or a combination thereof.

In certain embodiments, the combination the non-porcine lipase and the second active agent are each independently immediate release, delayed release, sustained release or a combination thereof.

In certain embodiments, the non-porcine lipase is a triacylglycerol hydrolase.

In certain embodiments, the non-porcine lipase has a molecular weight of about 30 to about 45 kDa, e.g., 37 kDa. In alternative embodiments, the non-porcine lipase contains from about 295 to about 310 amino acids, e.g., 301 amino acids.

In certain embodiments, the non-porcine lipase is produced from Yarrowia lipolytica. In alternative embodiments, the non-porcine lipase is encoded by the Lip2 gene. In a particular embodiment, the non-porcine lipase is MS1819.

In certain embodiments, the dosage forms disclosed herein are contained in a capsule wherein the capsule optionally includes an enteric material, e.g., coated over the capsule or dispersed within the capsule.

In certain embodiments, the dosing of the MS1819 is from about 1 g per day to about 10 g per day, about 2 g per day to about 5 g per day or about 2 g per day to about 4 g per day. In certain embodiments, the dosing is about 2.2 g per day or about 4.4 g per day.

In certain embodiments, the dosage forms disclosed herein comprise a tablet optionally comprising an enteric material, e.g., coated over the tablet or dispersed within the tablet.

In certain embodiments, the non-porcine lipase can be in the form of a powder optionally including an enteric material, e.g., by dry mixing, wet granulation or co-spray dried or co-freeze dried.

In certain embodiments, the formulation is in a liquid form, wherein the non-porcine lipase is in solution or suspension in a medium (e.g., an aqueous, non-aqueous or mixed medium) optionally including an enteric material.

In certain embodiments, the formulation is a powder or particles and contained in a capsule, sachet or powder paper.

In certain embodiments, the enteric material comprises a naturally occurring material or a non-naturally occurring material.

In certain embodiments, the enteric material comprises a cellulosic material, an acrylic polymer, or a combination thereof.

In certain embodiments, the enteric material comprises hydroxypropylmethylcellulose acetate succinate.

In certain embodiments, the enteric material comprises methacrylic acid polymers, cellulose acetate phthalate polymers, hydroxypropylmethyl cellulose acetate succinate polymers, hydroxypropylmethyl cellulose phthalate polymers, polyvinyl acetate phthalate polymers or combinations thereof.

In certain embodiments, the enteric material comprises methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, shellac or combinations thereof.

In certain embodiments, the enteric material comprises hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate or combinations thereof.

In certain embodiments, the enteric material is insoluble or substantially insoluble at a pH of less than about 4, less than about 3 or less than about 2.

In certain embodiments, the enteric material does not crack, break or rupture at a pH of less than about 4, less than about 3 or less than about 2.

In certain embodiments, the enteric material is soluble or substantially soluble at a pH of greater than about 5, greater than about 5.5, greater than about 6, greater than about 7 or greater than about 8.

In certain embodiments, the enteric material cracks, breaks or ruptures at a pH of greater than about 5, greater than about 5.5, greater than about 6, greater than about 7 or greater than about 8.

In certain embodiments, the dosage forms disclosed herein release less than about 10%, less than about 5%, less than about 3% or less than about 1% non-porcine lipase at 30 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage forms release less than about 10%, less than about 5%, less than about 3% or less than about 1% non-porcine lipase at 60 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% non-porcine lipase at 90 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% non-porcine lipase at 120 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or both of the active agents at 30 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or both of the active agents at 60 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or both of the active agents at 90 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or both of the active agents at 120 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases at least about 75%, at least about 90%, at least about 95% or at least about 99% non-porcine lipase at 15 minutes when tested in 900 mL simulated intestinal (at one or more points of buffer pH greater than or equal to 5.5, e.g., 5.5 or 6.0) at 37° C. in a USP Apparatus II at 100 rpm with or without sinkers.

In certain embodiments, the dosage form releases at least about 75%, at least about 90%, at least about 95% or at least about 99% non-porcine lipase at 30 minutes when tested in 900 mL simulated intestinal (at one or more points of buffer pH greater than or equal to 5.5, e.g., 5.5 or 6.0) at 37° C. in a USP Apparatus II at 100 rpm with or without sinkers.

In certain embodiments, the dosage form releases at least about 75%, at least about 90%, at least about 95% or at least about 99% non-porcine lipase at 45 minutes when tested in 900 mL simulated intestinal (at one or more points of buffer pH greater than or equal to 5.5, e.g., 5.5 or 6.0) at 37° C. in a USP Apparatus II at 100 rpm with or without sinkers.

In certain embodiments, the dosage form releases at least about 75%, at least about 90%, at least about 95% or at least about 99% non-porcine lipase at 60 minutes when tested in 900 mL simulated intestinal (at one or more points of buffer pH greater than or equal to 5.5, e.g., 5.5 or 6.0) at 37° C. in a USP Apparatus II at 100 rpm with or without sinkers.

In certain embodiments, the dosage forms disclosed herein target release of the non-porcine lipase in the duodenum of a patient in need thereof.

In certain embodiments, the non-porcine lipase is prepared by a process comprising drying, such as freeze drying or spray drying.

In certain embodiments, the spray draying utilizes a stabilizer such as an oligosaccharide, e.g., maltodextrin.

In certain embodiments, the dried non-porcine lipase is in the form of a powder or particles. The particles can have a particle size, e.g. with a D50 of about 50 micron to about 150 micron, about 60 micron to about 120 micron, about 65 micron to about 85 micron or about 70 micron to about 82 micron.

In certain embodiments, the stabilizer is maltodextrin, xylan, mannan, fucoidan, galactomannan, chitosan, raffinose, stachyose, pectin, inulin, levan, graminan, and amylopectin, sucrose, lactulose, lactose, maltose, trehalose, cellobiose, nigerotriose, maltotriose, melezitose, maltotriulose, raffinose, kestose, arginine, glycine, CaCl₂ or mixtures thereof.

In certain embodiments, the ratio of active to stabilizer is about 1:5 to about 5:1; about 1:3 to about 3:1; about 1:2 to about 2:1; about 1:1 or about 1:2.

In certain embodiments, the spray drying is performed at a pH of about 3 to about 5, about 2 to about 7, about 4 or about 6.

In certain embodiments, the spray drying is performed at a temperature of greater than about 125° C., greater than about 150° C., or from about 100° C. to about 250° C. or about 150° C. to about 180° C. or about 155° C. to about 165° C.

In certain embodiments, the spray drying produces the non-porcine lipase at a yield of greater than about 80%, greater than about 90%, greater than about 95% or greater than about 99%.

In certain embodiments, the dosage forms disclosed herein can further comprise a second active agent such as a fat-soluble vitamin (e.g., vitamin A, D, E, K and combinations thereof), a protease, an amylase, a porcine pancreatic enzyme replacement, other non-porcine replacements, or a combination thereof. In alternative embodiments, the second active agent is pancrelipase, liprotamase or a combination thereof. In a certain embodiment, the second active agent is a combination of three enzymes: lipase, protease, and amylase.

In certain embodiments, the invention is directed to a method of treating exocrine pancreatic insufficiency comprising administering a dosage form as disclosed herein.

In certain embodiments the insufficiency can be caused by one or more of acute or chronic pancreatitis, cystic fibrosis, pancreatectomy (associated with or without cancer such as pancreatic cancer), age related, Shwachman-Diamond Syndrome, diabetes type 1, diabetes type 2, HIV, celiac disease, or inflammatory bowel disease (such as ulcerative colitis or Crohn's disease).

In certain embodiments, the methods of treatment are solely with the non-porcine lipase formulations disclosed herein without the concurrent administration of a second active agent such as a fat-soluble vitamin (e.g., vitamin A, D, E, K and combinations thereof), a protease, an amylase, a porcine pancreatic enzyme replacement, other non-porcine replacements, pancrelipase, liprotamase a combination of three enzymes: lipase, protease, and amylase or a combination thereof.

In certain embodiments, the dosage form is administered by feeding tube in the form of a solution or suspension or sparkled on food in the form of a powder or administered as an oral dosage form such as a capsule, powder, tablet, liquid or semi-solid.

In certain methods disclosed herein, at least a portion of the non-porcine lipase is delivered to the duodenum of the patient. The portion can be, e.g., at least about 75%, at least about 85%, or at least about 95%.

In certain embodiments, the present formulations and methods provide a CFA % in individual patients or subjects from about 80 to about 92, about 85 to about 92, about 86 to about 92 or about 90 to about 92.

In certain embodiments, the present formulations and methods provide a CNA % in individual patients or subjects from about 90 to about 99, about 92 to about 99, about 95 to about 99 or about 99 to about 99.

In certain embodiments, the present formulations and methods provide a CNA % in a population of patients or subjects from about 90 to about 99, about 92 to about 99, about 95 to about 99 or about 99 to about 99.

In certain embodiments, the present formulations and methods when combined with a PPE provide a CFA gain relative to mean of about 3% to about 12%, from about 4% to about 10%, about 2% to about 6%, about 3% to about 6%, about 4%, about 5% or about 6%.

In certain embodiments, the present formulations and methods when combined with a PPE provide a maximum individual relative CFA gain from about 5% to about 50%, about 10% to about 45%, about 15% to about 40%, about 20% to about 50%, about 30% to about 40%, about 30%, about 35% or about 40%.

In certain embodiments, the invention is directed to preparing the compositions and formulations disclosed herein.

In certain embodiments, the non-porcine lipase is the secreted acid-resistant lipase (LIP2) from the yeast Yarrowia lipolytica. It belongs to the family of triacylglycerol lipases. It shares the common fold of a/b hydrolases and the crystal structure has been solved.

LIP2 is a 301 amino acid protein, which is secreted in culture medium as a glycosylated mature form after cleavage of a 39 amino acid signal peptide. Alternative cleavages have been evidenced on the lipase resulting in N-terminal sequence heterogeneity. The main N-terminal sequence was identified as STETSHIDQESYNFF in the spray-dried powder.

Two N-glycosylation sites have been identified at residues N113 and N134 and analysis by mass spectrometry has revealed that each site 1 harbors the following sugar moieties GlcNAc2-Manx (x=8). Five major glycoforms have been evidenced by isoelectrofocusing gels (IEF). In certain embodiments, the drug substance is defined as the spray-dried active agent bulk solution following the addition of Glucidex 12 in a ratio of 2:1 (based on bulk dry matter weight).

The sequence is as follows (SEQ ID NO.1):

VYTSTETSHIDQESYNFFEKYARLANIGYCVGPGTKIFKPFNCGLQCAH
FPNVELIEEFHDPRLIFDVSGYLAVDHASKQIYLVIRGTHSLEDVITDI
RIMQAPLTNFDLAANISSTATCDDCLVHNGFIQSYNNTYNQIGPKLDSV
IEQYPDYQIAVTGHSLGGAAALLFGINLKVNGHDPLVVTLGQPIVGNAG
FANWVDKLFFGQENPDVSKVSKDRKLYRITHRGDIVPQVPFWDGYQHCS
GEVFIDWPLIHPPLSNVVMCQGQSNKQCSAGNTLLQQVNVIGNHLQYFV
TEGVCGI

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1

Formulation of MS1819 Enteric Coated Capsules were prepared in accordance with Table 1.A.

TABLE 1.A

| Strength | | | 280 mg - |
|---|---|---|---|
| | | | 420,000 TBU |
| Batch Lot Number | | | 200749 |
| | Weight (kg) | 12.675 | MS1819 |
| | | 11.660 | MCC |
| | | 0.760 | Sodium starch |
| | | | Glycolate |
| | | 0.250 | Mg Stearate |
| | | 25.345 | Total Batch |

| | |
|---|---|
| Batch size (capsules) | 43,602 |
| Capsule | Size 0 DR |

The method of preparation was in accordance with the following process.

Step 1: Microcrystalline cellulose (MCC) is sieved through a rotating sieve with 1.0 mm mesh size screen and divided in two portions. Half of the microcrystalline cellulose was added into a 60 L drum. The MS1819 is sieved through a rotating sieve with 1.0 mm mesh size screen and introduced into the drum containing microcrystalline cellulose. The remaining microcrystalline cellulose is added into the drum containing microcrystalline cellulose and MS1819. Sodium starch glycolate is directly screened through a rotating sieve with 1.0 mm mesh size screen, into stainless steel drum containing microcrystalline cellulose and MS1819.

Step 2: The three materials are mixed for 10 minutes at 10 rpm.

Step 3: The magnesium stearate is directly sieved through a rotating sieve with 1.0 mm mesh size screen, into the drum containing pre-blend.

Step 4: The blend is mixed for 6 minutes at 10 rpm.

Step 5: Using a fully instrumented rotary tablet press, the final blend is compressed with a 19 mm tooling.

Step 6: Tablets are then milled through a rotating sieve with 1.5 mm mesh size grating screen.

Step 7: Magnesium stearate is weighed, sieved through a rotating sieve with 1.0 mm mesh size screen and transferred into the stainless steel drum containing the blend.

Step 8: The drum is blended at 10 rpm during 10 minutes with a bin blender.

Step 9: Using a fully instrumented encapsulator, the final blend is filled into hard capsules at the theoretical mass of 560 mg. The hard capsules are enteric and contain hydroxypropylmethyl cellulose.

Step 10: The capsules are sorted by a weight sorter with a mass tolerance of ±5% of theoretical mass.

### Example 2

A clinical trial was conducted to assess the safety and efficacy of MS1819 Enteric Capsules of Example 1 in patients with cystic fibrosis

The study, conducted under an IND and NCT No. NCT04375878, is a Phase 2, open-label, multicenter, 2×2 crossover trial to assess the safety and efficacy of MS1919 in enteric capsules in ˜30 patients with EPI due to CF.

The primary objectives of this study were to assess the safety and efficacy of MS1819 in enteric capsules vs porcine pancreatic enzyme replacement therapy (PERT) in patients with exocrine pancreatic insufficiency (EPI) due to cystic fibrosis (CF).

There were two arms and patients were randomized to participate in either the 2.2 g/day arm or 4.4 g/day arm of MS1819. The treatment duration on MS1819 was 3 weeks.

The formulation was safe and well-tolerated at all doses tested. The preliminary results for CFA % (n=24) are mean CFA % of about 59, min CFA % of 24 and max CFA % of about 92% with the number of patients above 80% being 5 and the preliminary results for CNA % (n=24) are mean CNA % of about 93, min CNA % of 83 and max CNA % of 99 with the number of patients above 90 being 22. The data showed a lack of dose response relationship.

The coefficient of fat absorption (CFA) and the coefficient of nitrogen absorption (CNA) for PERT is typically about 86 and 97 respectively.

As demonstrated in Example 2, the CFA for the present formulations approached and exceeded this value at with certain individual subjects.

As further demonstrated Example 2, the mean CNA for the population of subjects was about 93. This is surprising as it demonstrates that solely a lipase is sufficient to treat EPI without the need for a supplemental protease (although the two agents may still be combined).

### Example 3

The dissolution experimental setup used is similar but miniaturized to dissolution apparatus 2 (paddle) as described in USP <711>.

Buffers used were sodium Acetate 20 mM for pH ranging from 2.0 to 4.0 and IVIES 20 mM for pH ranging from 5.0 to 6.0.

A jacketed vessel, regulated at 37° C., was filled with 150 ml of buffer (pH 2 to 6) with or without pepsin (0.1 mg/ml). Stirring was performed using a magnetic stirrer set at reasonably low stir speed (level 2). At t=0, a capsule was weighted and then placed in a holder/sinker.

At 15, 30, 45 and 60 min time points, 20 μl samples were taken out for assay. At t=60′, pH was switched to 6 with the addition of NaOH 1N.

At time point 105 min (45 min after buffer change) 20 μl was taken out for assay

For analysis, 20 μl of dissolution medium was mixed with 5 μl of 5× loading buffer.

Three standard lipase DS samples were prepared at 2, 5 and 10 μg respectively by mixing 2, 5 and 10 μl of a 1 mg/ml C159001 solution in phosphate buffer (50 mM Na2HPO4 pH=6.0) to 5 μl of 5× loading buffer and elution buffer was added to get 25 μl final volume.

Samples (25 μl) were run at 300V for 18 minutes using the TGX stainfree AnyKDa Miniprotean 10 wells, 50 μl/well. Imaging was performed on the GelDoc EZ from BIORAD on a stain-free plate according to BIORAD manufacturing instructions.

For the dissolution of technical batch 20P002, the simulated gastric fluid (SGF) and simulated intestinal fluid (SIF) were prepared as an adaptation from the Minekus et al. paper. 100 ml stocks of the following buffers were prepared and combined as indicated in the below Table 3.A.

TABLE 3.A
Description of GSF pH = 3 and ISF pH = 6 compositions.

| Compound | [.](M) | V (ml) for 500 ml SGF | V (ml) for 500 ml SIF |
|---|---|---|---|
| KCl | 0.5 | 6.9 | 6.8 |
| KH₂PO₄ | 0.5 | 0.9 | 0.8 |
| NaHCO₃ | 1 | 12.5 | 42.5 |
| NaCl | 2 | 11.8 | 9.6 |
| MgCl₂, 6H₂O | 0.15 | 0.4 | 1.1 |
| (NH₄)₂CO₃ | 0.5 | 0.5 | — |
| CaCl₂, 2H₂O | 0.3 | 0.25 | 1 |
| | HCl 6N | 1.3 | 1 |
| | H₂O | qsp 500 ml | qsp 500 ml |

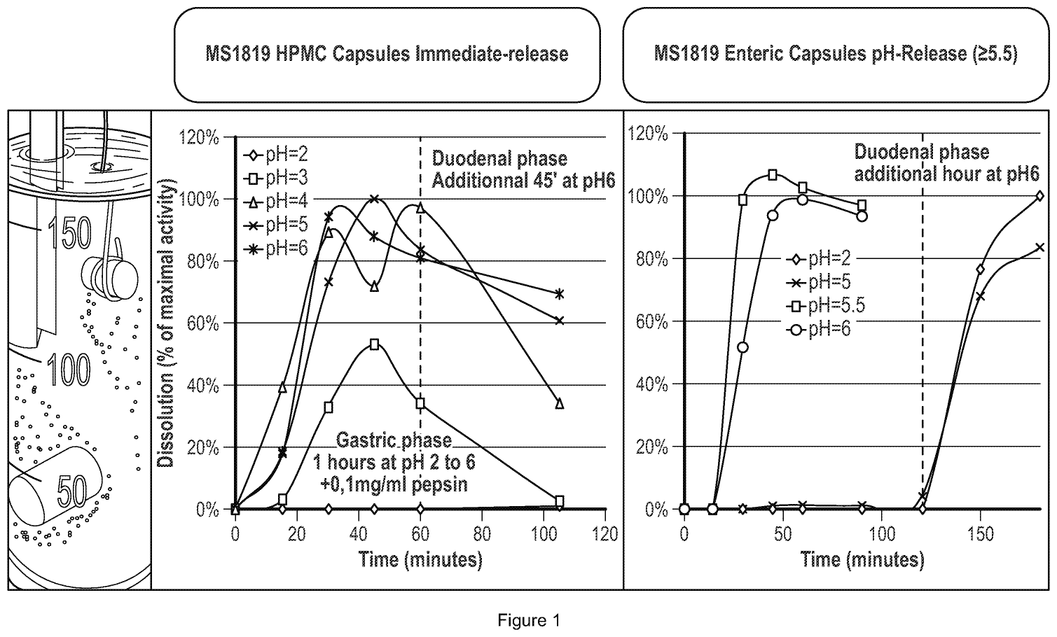

The results are set forth in FIG. 1. As demonstrated the immediate release formulation are not protected at low pH. In comparison, the enteric capsule formulation was protected at low pH and did not dissolve at a pH, e.g., less than or equal to 5.5. Therefore, the enteric capsule formulation may deliver higher amounts of the enzyme to the duodenum to digest fats.

### Example 4

A clinical combination study was performed under NCT No. NCT04302662 with the following protocol and the results are set forth in Table 4.A

The study is a multicenter, open-label Phase 2 study with escalating doses of MS1819-SD (spray dried MS1819 in an immediate release capsule) on top of a stable dose of PPEs, to investigate the efficacy and safety of this combination for the compensation of severe exocrine pancreatic insufficiency in CF patients not fully compensated with only PPEs.

The primary efficacy objective is to determine the efficacy of escalating doses of MS1819-SD on top of a stable dose of Porcine Pancreatic Extracts (PPEs) on triglyceride digestion assessed by coefficient of fat absorption (CFA) in patients with severe Exocrine Pancreatic Insufficiency (EPI) caused by Cystic Fibrosis (CF) and not fully compensated with only PPEs.

The primary safety objective is to assess the safety and tolerability of escalating doses of MS1819-SD on top of a stable dose of PPEs in patients with severe EPI caused by CF.

The design is multi-center, open-label, interventional study conducted in male and female patients with severe EPI caused by CF to investigate the safety and efficacy of escalating doses of MS1819-SD on top a stable dose of PPEs.

The study is conducted in 4 separate phases:

Phase A: Screening
All potential patients will be informed of study objectives and procedures.
Patients must be willing to provide written informed consent to participate in the study.
Patients affected by CF will be checked for all eligibility criteria.
Only patients treated with stable doses of PPEs for ≥1 months can participate in the study. Stable dose is defined as dose of medication not changed during this time period and the medication must be commercially available and be administered in the recommended dose range.
Complete physical examination is be performed.
Review medical history.
Specific assessment for CF (including a pulmonary function test by spirometry assessment to determine forced expiratory volume in 1 second [FEV1]≥30% of predicted normal for age, sex and height at screening and a sweat chloride test).
Measurement of fecal human pancreatic elastase-1 (FE-1) concentration within the last month is requested. If not available, stool samples for the measurement of FE-1 concentration are provided at screening visit. Only patients having a fecal human pancreatic elastase-1 concentration <100 μg/g of stools are proposed to move forward to phase B.

Phase B: Baseline with Coefficient of Fat Absorption (CFA) Measurement Under Routine Stable Dose of Porcine Pancreatic Extracts (PPEs) and Inclusion
After appearance of fecal stool dye markers, patients CFA are measured at inpatient facilities under their standard dose of PPE on standardized high-fat meals for 72 h.
The first dye marker is given to the participants with the first high fat meal and a second dye marker will be given at the end of the 72 h high fat diet period.
The first stool containing dye is discarded. Then stools are collected up to and including the first stool marked by the second dye marker.
The CFA calculation is based on the measured fecal fat content in relation to ingested fat quantities during a 72-hour time period. The ingested fat quantity per day is corrected to the real amount by subtracting residual food amount per meal/snack using a diet assessment. Average fat intake over the 72-hour stool collection period of less or more than 85-115 g fat per 24 hours of the planned amount makes the CFA assay invalid for the per protocol analysis. Only patients having a CFA <80%, with a maximum daily dose of 10,000 lipase units/kg/day according to US and European guidelines, move forward to phase C.
A minimum protein intake of 1.5 to 2 g/kg/day will be provided in the diet planned by a dietitian with the objective to assess CNA.

Phase C: Open-Label Escalating Dose of MS1819-SD (Cycles 1 to 3)
Routine PPEs (e.g., Creon® (pancrelipase) or Zempep® (pancrelipase)) are continued during the entire Phase C.
Patients receive increasing doses of MS1819-SD using the following 3 dose regimens during 15 days (±2 days) for each dose regimen: 700 mg/day, 1120 mg/day and 2240 mg/day.
Doses are fractionated as follows:
   700 mg/day: 1 capsule of 140 mg at each 3 main meals and at each of the 2 snacks
   1120 mg/day: 2 capsules of 140 mg at each 3 main meals and 1 capsule at each of the 2 snacks
   2240 mg/day: 4 capsules of 140 mg at each 3 main meals and 2 capsules at each of the 2 snacks
Each dose of MS1819-SD is administered from the start of each dose escalation for the entire planned 15-day (±2 days) period of the dose regimen and until the next visit.
The CFA and CNA are measured at inpatient facilities under MS1819-SD treatment and on standardized high fat meals for 72 h.
Uptitration to the next dose regimen in each subject are allowed following thorough review of the safety and tolerability of the preceding dose.
Total treatment duration with MS1819-SD (any dose) is a maximum of 51 days.

Phase D: Follow-Up/Early Termination
Standard PPE treatment is continued after MS1819-SD intake completion
A follow-up visit is scheduled after 12-15 days from end of Phase C.

Patients receive increasing doses of MS1819-SD as per following escalating ranges: 700 mg/day, 1120 mg/day and 2240 mg/day.

MS1819-SD is supplied as capsules of 140 mg each to be administered orally and taken with food.

The estimated study duration is approximately 90 days (including up to 15 days for Phase A (Screening period), 15 days for the Phase B (CFA measurement under routine stable dose of PPEs and inclusion) followed by a 15±2-day treatment period for each Cycle 1-3 in Phase C (open-label escalating dose of MS1819-SD), and 12-15 days in Phase D (Follow-up).

To be eligible for study entry patients must satisfy all of the following criteria:
1. Signed and dated informed consent form.
2. Age of 12 years at the time of screening
3. Male or female.
4. Under stable dose of PPE ≥1 month. Stable dose is defined as dose of medication not changed during this time period and the medication must be commercially available and be administered in the recommended dose range.
5. A nutritional status as defined by:
   a. BMI ≤22.0 kg/m2 for female patients
   b. BMI ≤23.0 kg/m2 for male patients
   c. BMI ≤50th percentile for patients 12 to <18 years of age.
6. Cystic fibrosis (CF), based on at least 2 clinical features consistent with CF in the opinion of the investigator and a sweat chloride concentration >60 mmol/L by pilocarpine iontophoresis.
7. Fecal pancreatic elastase-1<100 μg/g of stools at screening.
8. Baseline CFA <80% with a maximum daily dose of 10,000 lipase units/kg/day.
9. Clinically stable with no documented evidence of significant respiratory symptoms that would require administration of intravenous antibiotics, oxygen supplementation, or hospitalization within the 30 days of screening.
10. Male and female patients, if of childbearing potential, must use a reliable method of contraception during the study. A reliable method of birth control is defined as one of the following: oral or injectable contraceptives, intrauterine device, contraceptive implants, tubal ligation, hysterectomy, or a double-barrier method (diaphragm with spermicidal foam or jelly, or a condom), abstinence or vasectomy. Periodic abstinence (calendar, symptom-thermal, or post-ovulation methods) is not an acceptable method of contraception. The preferred and usual lifestyle of the patient must also be evaluated in determining if sexual abstinence is a reliable method of birth control.
11. Be considered as reliable and capable of adhering to the protocol, according to the judgment of the investigator.

Exclusion criteria is as follows:
1. Established or suspected fibrosing colonopathy.
2. Total or partial gastrectomy.
3. A history of solid organ transplant or significant surgical resection of the bowel; significant resection of the bowel is defined as any resection of the terminal ileum or ileocecal valve. Patients who have had qualitative, long-term changes in nutritional status after any other bowel resection (e.g., increased of new need for pancreatic enzyme supplementation compared with preoperative status to maintain the same nutritional status) should also be excluded.
4. Any chronic diarrheal illness unrelated to pancreatic insufficiency (e.g., infectious gastroenteritis, sprue, inflammatory bowel disease)
5. Known hypersensitivity or other severe reaction to any ingredient of the investigational medicinal product (IMP).
6. Bilirubin >1.5 times upper limit normal (ULN).
7. Alanine aminotransferase (ALT) or aspartate aminotransferase (AST)>5 times ULN.
8. Alkaline phosphatase (ALP)>5 times ULN.
9. Gamma glutamyltransferase (GGT)>5 times ULN.
10. Signs and/or symptoms of liver cirrhosis or portal hypertension (e.g., splenomegaly, ascites, esophageal varices), or documented liver disease unrelated to CF
11. Known allergy to the stool marker.
12. Feeding via an enteral tube during 6 months before screening
13. Routine use of anti-diarrheals, anti-spasmodics, or cathartic laxatives, or a change in chronic osmotic laxatives (e.g., polyethylene glycol) regimen in the previous laxative therapy within the last 12 months before screening
14. History of severe constipation with <1 evacuation/week under appropriate laxative therapy within the last 12 months before screening.
15. Documentation of distal intestinal pseudo-obstruction syndrome within the last 12 months before screening.
16. Forced Expiratory Volume ≤30% at the screening visit.
17. Lactation or known pregnancy or positive pregnancy test at both screening and baseline for women of childbearing potential.
18. Participation in another clinical study involving an IMP within 30 days before inclusion or concomitantly with this study.
19. Poorly controlled diabetes according the investigator's judgement.

Standard-of-care medications for CF such as antibiotics, mucolytic agents, aerosols and CFTR modifiers are allowed. CFTR modulators should be on stable doses for at least 3 months. Patients should not start taking CFTR modulators during the duration of the study.

Gastric acid suppressants are allowed but must be on stable dosage for 30 days before screening and must not be altered in dose or stopped during the study.

Prohibited medications are as follows:
Orlistat lipase inhibitor (e.g., AlliR®, Xenical®),
Laxatives consisting of mineral oil and castor oil (chronic use of osmotic laxatives is permitted)
Symptomatic treatments of diarrhea: loperamide (loperamide generic, Imodium®, Imodium A-D®, Diamode®, Imotil®, Kao-Paverin®), atropine/diphenoxylate (Lonox®), atropine/diphenoxylate (Lomocot®).

The primary efficacy endpoint is as follows:
Change in CFA from baseline (V2) to visits of phase C (V4, V5 and V6).

Secondary endpoints are as follows:

### Key Secondary Endpoints:

Change from Phase B in the mean number of daily evacuations during the days of the stool collection period in each cycle in Phase C.
Change from Phase B in the mean consistency of stools assessed by the Bristol scale during the stool collection period in each cycle in Phase C.

### Other Secondary Endpoints:

Body weight.
Body mass index.
Weight of stools during the stool collection period (per 24 hours and over a 72-hour collection period).
Abdominal discomfort assessed by visual analogue scale.
Absorption variables: CNA and steatorrhea.
Change from Phase B in the mean number of daily evacuations per day over each full cycle in Phase C.
Change from Phase B in the mean consistency of stools assessed by the Bristol scale over each full cycle in Phase C.

Safety data, including all observed AEs with a particular focus on immunoallergic events and digestive symptomatology.

Secondary safety endpoints are as follows:

In addition to primary safety endpoints, laboratory test results will be summarized:
Fasting glucose.
Urinalysis
Hematology: Hematocrit, Hemoglobin, Erythrocyte count (RBC), Leukocytes (WBC), Absolute counts of: Neutrophils (segmented), Neutrophils juvenile (bands), Lymphocytes, Monocytes, Eosinophils, Basophils and Platelets.
Biochemistry: Serum concentration of: Sodium, Potassium, Chloride, Bicarbonate, Blood urea nitrogen (BUN), Total Calcium, Phosphorus, Magnesium, Albumin, Prealbumin, Total protein, Creatinine, Alkaline phosphatase, Alanine aminotransferase (ALT), Aspartate aminotransferase (AST), Lactate deshydrogenase (LDH), Total bilirubin, Direct bilirubin, Uric Acid.
Fasting Lipid Profile: Total cholesterol, Triglycerides, Low Density Lipoprotein (LDL), High Density Lipoprotein (HDL) and Very Low Density Lipoprotein (VLDL)
Serum vitamins A, D, E and K.
Activated partial thromboplastin time (aPTT), Prothrombin time/International normalized ratio (PT/INR)
Immunogenic assessment for circulating levels of LIP2 lipase.
Antibodies against LIP2.

The sample size determination is based on the efficacy primary endpoint, i.e. the change baseline in CFA from baseline (V2) to visits V4, V5 and V6 of the phase C.

Assuming a standard deviation for the change in CFA of 15%, an expected mean CFA change of 10% for at least one dose of MS1819-SD (700 mg/d, 1120 mg/d or 2240 mg/d), a two-sided nominal alpha level of 0.05, 20 patients are sufficient to achieve a power of 80%. Accounting for possible drop-outs during dose escalation, 24 patients will be enrolled.

24 patients are considered enough to adequately assess the safety of each studied dose.

As there is some uncertainty about the expected standard deviation for the change in CFA in the population studied, the standard deviation will be re-estimated at an interim stage (after 15 patients have completed the phase C). If the estimated standard deviation is larger than the planned one, the sample size will be increased to maintain the power.

Efficacy Analysis Sets
Full analysis set (FAS): is defined as all patients receiving at least 1 dose of treatment and with some efficacy assessment available on treatment. The FAS is considered the primary set for the efficacy analysis.
Per-Protocol set (PP): is a subset of the FAS comprising all patients who do not violate the terms of the protocol in a way that would affect the study outcome significantly, as determined by the study clinician.

Safety Set:
The safety set is defined as all patients who receive at least 1 dose of MS1819-SD.
Patients will be analyzed according to the treatment actually received.

### Efficacy Analysis:

Primary Efficacy Endpoint:

The primary analysis will be performed on the FAS. The primary efficacy endpoint (change of CFA during Phase C) will be analyzed in a mixed model for repeated measures (MMRM) including a random term for patient, a fixed term for visits and the baseline CFA as a covariate. Estimation will be performed using the Restricted Maximum Likelihood (REML) approach under the assumption of an unstructured covariance matrix for repeated measurements. The mean change of CFA from baseline to each escalated dose visit will be estimated along with its 95% Confidence Interval and p-value assuming with the baseline CFA set to its mean level estimated at baseline.

Sensitivity Analyses of the Primary Efficacy Endpoint
The primary efficacy endpoint will be analyzed at each visit in an Analysis of Covariance (ANCOVA) Model including an intercept and the baseline CFA. Missing data will not be replaced.
This ANCOVA analysis will also be performed with missing data replaced by the Las Observation Carried Forward (LOCF) method.

Dose Response Modelling

A random coefficient model will be fitted to assess the dose response relationship. The dose taken during each escalating period will be included as linear and quadratic (dose squared) fixed covariates. This model will also include random terms for intercept, linear and quadratic trends of dose assuming an unstructured covariance matrix for these parameters. If convergence issues occur when fitting the model, the linear trend will only be kept in the model.

Subgroup Analyses

Analyses will be performed in the following subgroups:
Age (<18 vs ≥18 years)
Use of PPIs (Yes vs no)

Key Secondary Efficacy Endpoints:
Change from baseline in the mean number of daily evacuations during the days of the stool collection period in each escalated dose period of Phase C
Change from baseline in the mean consistency of stools assessed by the Bristol scale during the stool collection period in each escalated dose period of Phase C

Other Secondary Endpoints Analyses:

The analysis of the other secondary efficacy endpoints will be detailed in the statistical analysis plan.

Safety Analyses:
Exposure to study drug and reasons for discontinuation or withdrawal will be tabulated.
Safety will be evaluated by the incidence of AEs, severity and type of AEs, and by changes from baseline (Phase B) in the patient's vital signs, weight, and clinical laboratory results using the safety population.

The formulation was safe and well-tolerated at all doses tested. The preliminary results (n=18) show a mean gain relative to baseline of about 5.9% with the max gain relative to baseline of 34 and the min gain relative to baseline of 16 and the number of above 80% being 7. The data showed a lack of dose response relationship.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

Reference throughout this specification to numerical ranges should not be construed as limiting and should be understood as encompassing the outer limits of the range as well as each number and/or narrower range within the enumerated numerical range.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims

1. A delayed release oral dosage form comprising:
(a) a non-porcine lipase; and
(b) an enteric material encompassing the non-porcine lipase.

2. The delayed release oral dosage form of claim 1, wherein the non-porcine lipase is a triacylglycerol hydrolase.

3. The delayed release oral dosage form of claim 1, wherein the non-porcine lipase has a molecular weight of about 30 kDa to about 45 kDa.

4. The delayed release oral dosage form of claim 1, wherein the non-porcine lipase has a molecular weight of about 37 kDa.

5. The delayed release oral dosage form of claim 1, wherein the non-porcine lipase contains from about 295 to about 310 amino acids.

6. The delayed release oral dosage form of claim 1, wherein the non-porcine lipase contains about 301 amino acids.

7. The delayed release oral dosage form of claim 1, wherein the non-porcine lipase is produced from Yarrowia lipolytica.

8. The delayed release oral dosage form of claim 1, wherein the non-porcine lipase is encoded by the Lip2 gene.

9. The delayed release oral dosage form of claim 1, wherein the non-porcine lipase is MS1819.

10. The delayed release oral dosage form of claim 1, comprising a capsule containing the non-porcine lipase, wherein the capsule comprises the enteric material, e.g., as a coating or dispersed within the capsule.

11. The delayed release oral dosage form of claim 1, comprising a tablet comprising the non-porcine lipase, wherein the tablet is overcoated with the enteric material.

12. The delayed release oral dosage form of claim 1, wherein the non-porcine lipase is combined with the enteric material in a solution and freeze dried or spray dried to form a powder.

13. The delayed release oral dosage form of claim 1, wherein the non-porcine lipase is in solution or suspension in a medium.

14. The delayed release oral dosage form of claim 1, wherein the powder is contained in a capsule, sachet or powder paper.

15. The delayed release oral dosage form of any preceding claim, wherein the enteric material comprises a naturally occurring material or a non-naturally occurring material.

16-49. (canceled)

50. A method of treating exocrine pancreatic insufficiency comprising administering a delayed release dosage form according to claim 1.

51. A method of treating acute or chronic pancreatitis comprising administering a delayed release dosage form according to claim 1.

52. A method of treating cystic fibrosis comprising administering a delayed release dosage form according to claim 1.

53-59. (canceled)

60. A process of preparing a delayed release oral dosage form comprising combining a non-porcine lipase and an enteric material to form a dosage form according to claim 1.

61. A dosage form comprising:
(a) a non-porcine lipase; and
(b) a second active agent selected from a fat-soluble vitamin, a protease, an amylase, a porcine pancreatic enzyme replacement, other non-porcine replacements, or a combination thereof.

62-121. (canceled)
